(19) United States
(12) Patent Application Publication
(10) Pub. No.: US 2025/0087347 A1
(43) Pub. Date: Mar. 13, 2025

(54) ARTIFICIAL INTELLIGENCE-BASED SYSTEM FOR MONITORING PATIENTS FACILITATING IN MEDICATION ADHERENCE AND METHOD THEREOF

(71) Applicants: Rishi V, Frisco, TX (US); Venkatesh Cumbakonam Gokulraju, Frisco, TX (US)

(72) Inventors: Rishi V, Frisco, TX (US); Venkatesh Cumbakonam Gokulraju, Frisco, TX (US)

(21) Appl. No.: 18/757,550

(22) Filed: Jun. 28, 2024

(51) Int. Cl. G16H 40/67; G06F 21/31; G16H 20/13

(52) U.S. Cl. CPC G16H 40/67; G06F 21/316; G16H 20/13

(57) ABSTRACT

An artificial intelligence (AI) system for monitoring a patient and facilitating in medication adherence for users is disclosed. The system comprises a patient monitoring device, a user device, and one or more biomedical devices. The patient monitoring device is in communication with the user device and the biomedical devices through a server. The patient monitoring device comprises a processor that is configured to execute a plurality of modules, which includes an input module, an AI processing module, a monitoring module, and alert and output modules. The system remotely monitors the patient's health in real-time and facilitate in the medication adherence for the users. The AI based system provides assistance to the patients in taking their medications as prescribed, which could include features such as reminders, notifications for taking medications on time and in the correct dosage.

The present disclosure relates generally to systems and methods for medication management, and more particularly to an artificial Intelligence (AI) based system for remotely monitoring patients in real-time and facilitating in medication adherence for users.

### BACKGROUND

Living standards and medical advancements have significantly extended human lifespans. However, this doesn't guarantee a cure for every illness. Even with medical attention, some conditions remain chronic or incurable. Treatment may focus on managing symptoms and preventing worsening with medication or therapies. Additionally, some patients might require short-term post-hospitalization care to support their recovery.

Certain illnesses demand strict adherence to multiple therapies or exercises each day. This might involve medications with a narrow window for effectiveness or safety. Patients who are dealing with memory-related issues, such as early dementia, or physical limitations, post-operative care requirements, chronic disease, and Alzheimer's disease, require special attention and care. These patients often need assistance with daily activities and medical management to maintain their health and wellbeing. These patients may rely on home care, often from family members or non-professionals. Home care settings typically lack sophisticated monitoring equipment, requiring trips to doctors' offices or diagnostic facilities for proper evaluation and treatment.

Patients with conditions like Alzheimer's disease, dementia, or Parkinson's disease often experience memory decline and executive dysfunction. This can make it difficult for them to recall medication schedules, dosing instructions, or even the purpose of the medication itself. Patients with severe physical limitations, such as those with multiple sclerosis or advanced arthritis, may struggle with the physical dexterity required to open medication bottles, manipulate tablets, or administer injections.

At present, delivery of healthcare is possible through electronic communication, or telehealth to overcome the abovementioned problems. Over the years, telehealth has rapidly evolved with the continued advancement in available technology and the innovations by the healthcare community in identifying new uses and applications for technology. The implementation of electronic health records (EHRs) and expanded access to the internet and medical devices have enabled healthcare to move outside of conventional clinical settings and into a patient's home.

Remote patient monitoring (RPM) is a form of telehealth where healthcare providers keep track of patients' health status outside of the conventional care setting by utilizing digital medical devices. Examples of these medical devices include weight scales, blood pressure monitors, pulse oximeters, and blood glucose meters. The data collected from these devices are then transferred electronically to healthcare providers for care management. Automated feedback and workflows can be incorporated into data collection, and any out-of-range values or concerning readings can be flagged. RPM is used to detect symptoms of chronic conditions like cardiac diseases and diabetes through wearable devices such as Holter monitors.

However, healthcare organizations struggle to effectively implement RPM systems. Training healthcare providers is difficult because conventional RPM systems require managing complex tasks. Additionally, integrating devices with existing RPM systems can be time-consuming and expensive due to custom coding and testing. The conventional RPM systems lack accuracy, comfort, and case of use for patients. The conventional RPM systems also fail to provide easy-to-use user interfaces that can translate raw data into clear and actionable insights. The raw data generated by the integrating devices can be overwhelming, and it requires a considerable amount of time and effort to interpret and analyze the data accurately. This can be a significant challenge for healthcare professionals who have to manage multiple patients and may not have the time to go through all the data manually. This leads to an increase in the time required to decipher complex data points for patient care.

Moreover, the conventional RPM systems are not very user-friendly and can be uncomfortable for patients. The devices are often bulky, and wearing them for extended periods can cause discomfort and irritation. Additionally, the accuracy of the results obtained from these systems is not always reliable, which can lead to incorrect diagnoses and treatment plans.

Therefore, there is a need for a system that remotely monitors patient's health in real-time continuously. There is also a need for a system that detects abnormality in a patient's behavior, physiological data, and patient's gestures to predict patient's real-time condition. There is also a need for a system that is integrated with a smart medicine dispensing device that facilitate the medication adherence. There is also a need for system that can observe activities of daily living (ADLs) to detect a person's functional status. There is also a need for a system that to provide accurate, comfortable, and easy-to-use devices that can generate clear and actionable insights for healthcare professionals.

### SUMMARY OF THE INVENTION

The following presents a simplified summary of one or more embodiments of the present disclosure to provide a basic understanding of such embodiments. This summary is not an extensive overview of all contemplated embodiments and is intended to neither identify key nor critical elements of all embodiments, nor delineate the scope of any or all embodiments.

The present disclosure, in one or more embodiments, relates to a system for dispensing medicine to a patient. In one embodiment herein, the system comprises a patient monitoring device, a user device, and one or more biomedical devices. The patient monitoring device is in communication with the user device and the biomedical devices through a server via a network.

In one embodiment, the patient monitoring device comprises a processor and a memory. The memory is configured for storing one or more instructions executable by the processor. The processor is configured to execute a plurality of modules. The modules comprises an input module, an artificial intelligence (AI) processing module, a monitoring module, an alert module, and an output module.

In one embodiment, the processor is configured to receive medical data of a patient and prescription data from authorized user through the input module via the user device. In one embodiment, the patient monitoring device is configured to deliver a dose to the patient based on the prescription data. The medical data comprises a patient name, age, patient medical history data, patient symptoms, patient vital sign data, and a laboratory test data. Based on the medical data, the authorized user is enabled to create a patient profile of the patient in the system. The authorized user comprises at least one of physicians, payers, caregivers, a pharmacist, guardian, doctor.

In one embodiment, the monitoring module is configured to receive physiological and behavioral data of the patient in real-time. In one embodiment, the monitoring module is configured to track the physiological and behavioral data of the patient through at least one of a camera, and the biomedical devices. In one embodiment, the biomedical devices that include at least one of digital thermometers, blood pressure monitors, pulse oximeters, blood glucose meters, nebulizers, canes, medical compression stockings, transcutaneous electrical nerve stimulation units (TENS), and breast pumps thereof.

In one embodiment, the monitoring module is configured to track schedule data. The schedule data comprises at least one of medication dosage, medication administration times, medication name, frequency of medication administration, route of administration, exercise periods, type of exercise, time duration for the exercise, and intensity of the exercise thereof.

In one embodiment, the artificial intelligence (AI) processing module is configured to analyze the medical data to detect baseline data of the patient. The artificial intelligence (AI) processing module is further configured to perform a predictive analysis on the physiological and behavioral data to identify activities of daily living (ADLs) of the patient. The artificial intelligence (AI) processing module is furthermore configured to detect abnormality actions in the physiological and behavioral data and the ADLs and generate insights and predictive analytics based on the analyzed the medical data and the ADLs.

In one embodiment, the artificial intelligence (AI) processing module is integrated with a gesture-detection module. The gesture-detection module is configured to perform predictive analysis to detect the ADLs based on the physiological and behavioral data of the patient. In one embodiment, the patient's abnormality is detected based on deviation in the physiological and behavioral data of the patient with respect to the baseline data. The physiological and behavioral data of the patient comprises a real-time patient vital sign data, facial expression, postures, gestures, limb positions, hand positions, reflexes, qualities of a patient's voice, and intensity.

In one embodiment, the alert module is configured to transmit an alert to the authorized user upon detection of the abnormality actions. In one embodiment, the alert module is configured to provide the alerts or reminders in a form of either audio or video or notification or call for missed medications or upcoming exercise sessions. The output module is configured to transmit the schedule data, and the generated insights and predictive analytics to at least one of the authorized user and the patient.

In one embodiment, the patient monitoring device comprises a plurality of holders that are mounted on both sides of the patient monitoring device, wherein each holder is configured holding at least one medicine. Each holder is configured with at least one sensor that is configured for at least one of an inventory control, a dosage verification, and detecting empty or missing the medicine. The camera of the monitoring module is configured to capture either video or image data of a patient during medication consumption activity. The captured video or image data is analyzed by the gesture-detection module to detect the abnormality actions that is associated with medication adherence. The abnormality actions are indicative of potential non-adherence behaviors. The gesture-detection module is configured to compare the captured video or image data with the schedule data to identify the timing and frequency of medication adherence events to identify the potential medication non-adherence, such as missed doses or potential overdoses, thereby provide the alerts or reminders in a form of at least one of audios, videos, notifications, and calls to the authorized person.

An embodiment of the first aspect wherein a method for monitoring patient's health in real-time and facilitate in medication adherence for users using the patient monitoring device. In first step, the method comprises, receiving physiological and behavioral data of the patient in real-time through the AI processing module from the monitoring module and the biomedical devices. The method comprises, analyzing medical data of the patient to identify baseline data of the patient through the AI processing module. The method comprises, performing a predictive analysis on the physiological and behavioral data through the AI processing module, and identifying activities of daily living (ADLs) of the patient through the gesture-detection module. The method comprises, detecting abnormality actions of the patient based on the physiological and behavioral data and the ADLs through the AI processing module, thereby generating insights and predictive analytic reports for the patient.

The method comprises, allowing the authorized person to enter the prescription data and the schedule data via the user interface of the patient monitoring device for creating a patient profile. The method comprises, dispensing the medications through the outlet of the patient monitoring device based on the prescription data, and the schedule data upon patient authentication by the gesture-detection module. The method comprises, identifying the one or more medications that are dispensed through the outlet via an identification module using the AI processing module. This ensures to dispense prescribed medication for the patient, which results in improving medication adherence. The method comprises, transmitting one or more alerts to the authorized user through the alert module upon detection of the abnormality actions of the patients.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1A illustrates a block diagram of an artificial intelligence-based medication dispensing system with real-time health monitoring and activity recognition, in accordance with embodiments of the invention.

FIG. 1B illustrates a perspective front view of a patient monitoring device, in accordance with embodiments of the invention.

FIG. 1C illustrates an exploded view of the patient monitoring device, in accordance with embodiments of the invention.

FIG. 2 illustrates a flowchart depicting a method for real-time health monitoring and activity recognition, in accordance with embodiments of the invention.

FIG. 3 illustrates a flowchart depicting a method for refilling medication in the patient monitoring device, in accordance with embodiments of the invention.

FIG. 4 illustrates a flowchart depicting a method for dispensing pill medication from the patient monitoring device, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

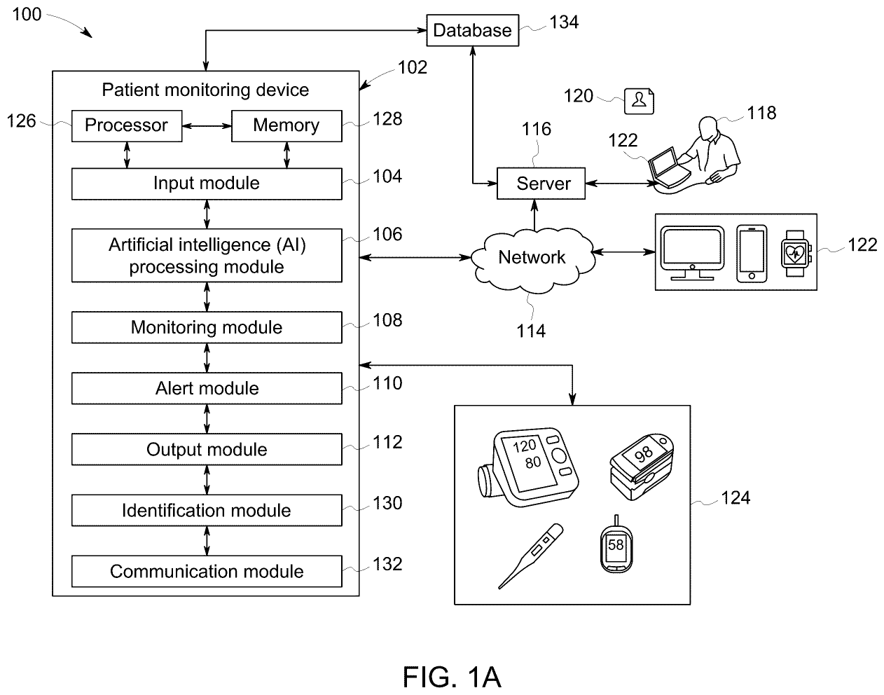

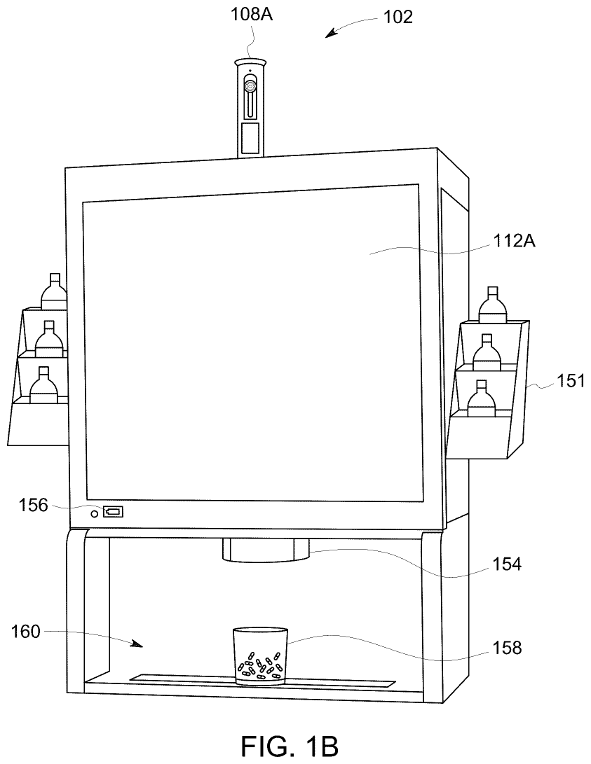

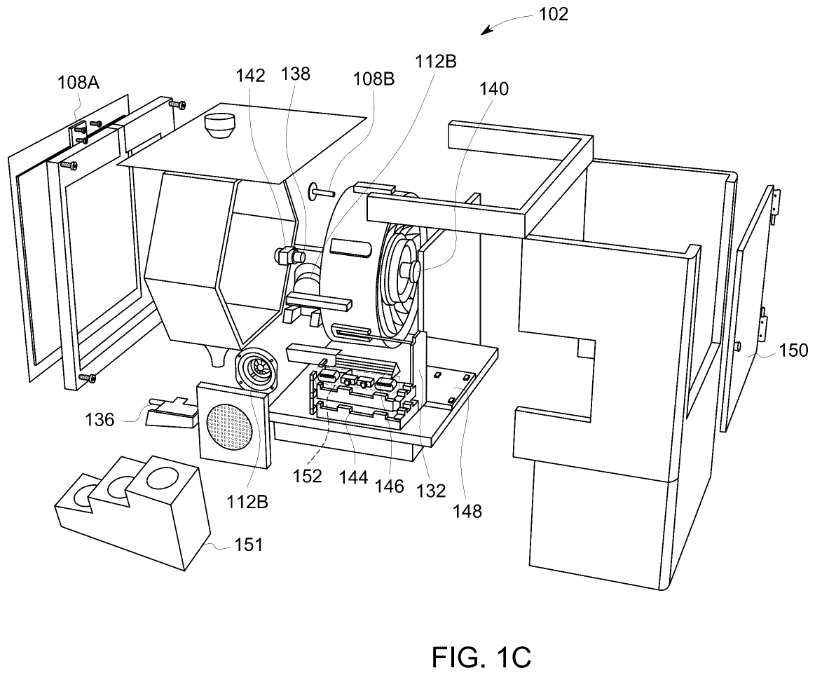

FIG. 1A refers to a block diagram of an artificial intelligence (AI) based system 100 for monitoring patients in real-time. FIG. 1B refers to a perspective view of a patient monitoring device 102. FIG. 1C refers to an exploded view of the patient monitoring device 102. In one embodiment herein, the AI based system 100 is configured to remotely monitor the patients in real-time and facilitate in medication adherence for users. The AI based system 100 could help patients achieve better health outcomes and avoid potential negative consequences of non-adherence, such as increased hospitalizations or disease progression. In one embodiment herein, the AI based system 100 comprises the patient monitoring device 102, a network 114, a server 116, a user device 122, one or more biomedical devices 124, and a database 134.

In one embodiment, the patient monitoring device 102 is in communication with the user device 122 and the biomedical devices 124 through the server 116 via the network 114 for data transferring. In another embodiment, the network 114 is a communications means of the AI based system 100. The network 114 may be any circuitry or other means for communicating data over one or more networks or to one or more peripheral devices attached to the AI based system 100. The network 114 may include, but are not limited to, cellular network, Wi-Fi, Bluetooth, near field communications (NFC), one or more long range (LoRA) modules, and any combination thereof.

Referring to FIG. 1A, the patient monitoring device 102 comprises a processor 126 and a memory 128. The memory 128 is configured for storing one or more instructions executable by the processor 126. The processor 126 is configured to execute a plurality of modules for control one or more operations performed by the AI based system 100. In one embodiment herein, the plurality of modules comprises an input module 104, an artificial intelligence (AI) processing module 106, a monitoring module 108, an alert module 110, and an output module 112, an identification module 130, and a communication module 132. In another embodiment, the input module 104, and the output module 112 act as a user interface.

In one embodiment herein, the processor 126 is configured to receive medical data of a patient, prescription data and schedule data from authorized user 118 through the input module 104 via the user interface of the patient monitoring device 102. The medical data comprises a patient image, a patient name, age, patient medical history data, patient symptoms, patient vital sign data, and a laboratory test data. Based on the medical data, a patient profile 120 is created by the authorized user 118 in the AI based system 100. The patient profile 120 is stored in at least one of in the server 116 or the database 134. The authorized user 118 comprises, but is not limited to, physicians, payers, a pharmacist, guardian, doctor, and a remote caregiver. In an exemplary embodiment, the authorized user 118 is the patient.

In another embodiment, the patient profile 120 is configured to store the prescription data, and update the prescription data and the medical data. The prescription data may include medications or treatments that authorized user 118 has authorized for the patient. As such, the server 116 sends the authorized prescription data to the connected patient monitoring device 102.

In one embodiment herein, the monitoring module 108 is configured to receive physiological and behavioral data of the patient in real-time. The monitoring module 108 is configured to track the physiological and behavioral data of the patient. In one embodiment herein, the monitoring module 108 could be, but not limited to, a camera 108A, an image sensor, a microphone 108B and any capturing unit or device, as shown in FIGS. 1B-1C. In one embodiment herein, the biomedical devices 124 include, but are not limited to, digital thermometers, blood pressure monitors, pulse oximeters, blood glucose meters, nebulizers, canes, medical compression stockings, transcutaneous electrical nerve stimulation units (TENS), and breast pumps thereof, as shown in FIG. 1A.

In another embodiment, the patient monitoring device 102 is communication with the biomedical devices 124 through an open source platform such as EdgeX platform. In another embodiment, the EdgeX platform comprises an edge core module, the edge core module comprises an edge hub module.

In another embodiment, the server 116 comprise at least one of an API-Server module, a kube-controller-manager module, an edge controller module, and a cloud hub module.

In an exemplary embodiment, the API-Server module is used for receiving a client request, and the kube-controller-manager module is used for controlling a control unit 156 of the AI based system 100. The edge controller module is used for synchronizing information of an edge node and a main control node module, controlling metadata of the edge node and the edge module and ensuring that the data can be transferred to a designated edge node; and the Cloud Hub module is communicated with the edge node based on protocol connection, monitors cloud changes and realizes cloud edge communication.

In one embodiment herein, the monitoring module 108 is configured to track the schedule data. The schedule data comprises, but is not limited to, medication dosage, medication administration times, medication name, frequency of medication administration, route of administration, exercise periods, type of exercise, time duration for the exercise, and intensity of the exercise, and thereof.

In one embodiment herein, the AI processing module 106 is configured to analyze the medical data to detect baseline data of the patient. The AI processing module 106 is further configured to perform a predictive analysis using a gesture-detection module on the physiological and behavioral data to identify activities of daily living (ADLs) of the patient. The AI processing module 106 is furthermore configured to detect abnormality actions in the physiological and behavioral data and the ADLs of the patient and generate insights and predictive analytics based on the analyzed the medical data and the ADLs.

In one embodiment herein, the gesture-detection module is configured to perform predictive analysis to detect the ADLs based on the physiological and behavioral data of the patient. In one embodiment herein, the patient's abnormality is detected based on deviation in the physiological and behavioral data of the patient with respect to the baseline data. The physiological and behavioral data of the patient comprises a real-time patient vital sign data, facial expression, postures, gestures, limb positions, hand positions, reflexes, qualities of a patient's voice, and intensity.

In another embodiment, the AI based system 100 is configured to facilitate in medication adherence for the patients and the users. In one embodiment herein, the AI processing module 106 is adapted to analyze the medical data received about the patient. This data may include name, age, gender, existing conditions, allergies, medications, heart rate, blood pressure, and thereof. Based on the patient's data, the AI processing module 106 establishes a baseline for the patient's health. This baseline serves as a reference point for identifying any potential abnormalities.

In one embodiment, the AI processing module 106 is configured to receive input from at least one of the monitoring module 108, for example, the camera 108A, or one or more microphones 108B, and thereof. The AI processing module 106 is in communication with the gesture-detection module for extract one or more image features from the image data, which can include images or videos of the user, the user's face, or other body parts associated with a medical condition.

In one embodiment, the monitoring module 108 is set up to remotely and continuously monitor patients in a variety of ways, from identifying patients in extreme distress to monitor patient's behaviors, such as hand-to-mouth motions when consuming medicine includes pills or syrup. In some embodiments herein, the monitoring module 108 could be, but not limited to, an artificial intelligence camera.

In one embodiment herein, the AI processing module 106 integrated with the gesture-detection module analyzes the postures, gestures, limb positions, hand positions to determine if the patient consumed the prescription medication. In another embodiment, the gesture-detection module may determine if the patient consumed the medication dose based on sensor input, including, for example, image analysis, audio analysis, motion detection, or by measuring patient biological response to the prescription medication dose.

Further, the gesture-detection module could also be a pre-trained facial recognition model that identifies the patients based on their facial features. In another embodiment, the patient profile 120 along with a patient's image could be stored in the server 116.

In one embodiment herein, the monitoring module 108 is configured utilize gait analysis to track and remotely monitor whether the patient is performing the prescribed exercises. This feature is crucial in ensuring that the patient stays on track and achieves their recovery goals in a timely and efficient manner. In one embodiment herein, the monitoring module 108 also provides real-time feedback to the healthcare provider, thereby enabling them to make informed decisions about the patient's treatment plan.

In one embodiment herein, the alert module 110 is configured to transmit one or more alerts to the authorized user 118 upon detection of the abnormality actions. In one embodiment herein, the alert module 110 is configured to provide the alerts or reminders in a form of at least one of, but not limited to, audios, videos, notifications, and calls for missed medications and upcoming exercise sessions.

In one embodiment, the alert module 110 is also configured to provide accurate feedback on patient's posture, alignment, and balance during preforming the prescribed exercises. The AI processing module 106 analyzes the captured image data received from the monitoring module 108 and evaluate the patient's posture, alignment, and balance based on standard exercise pose for detecting incorrect pose of the patient while performing the prescribed exercises.

In another embodiment, the AI based system 100 configured to create a pose landmark of the patient. The pose landmark is a set of specific points on the patient's body that are used to track the patient's posture, alignment, balance and movement. In one embodiment, the AI processing module 106 and the gesture-detection module are involved in creating the pose landmark from a video input.

For example, when the patient is performing any prescribed exercise. The camera 108A provides the video input of the area to detect and identify the patient. Then, the AI based system 100 uses the AI processing module 106 to find the patient in frames. If the patient is detected, a region of interest (ROI) is set up around the patient, which is used to focuses on relevant part of the image for pose estimation. In one embodiment herein, the AI based system 100 uses the gesture-detection module to find key-points on the patient's body, such as the elbows, knees, and shoulders. These key-points are then connected to create the pose landmark.

In another embodiment, the key points within the ROI are localized and detected. The key point detection is classified into a global body and a local body. It appears that 33 key points are identified in total.

In one embodiment herein, the AI based system 100 further includes an action monitoring function that can be used to track the patient's movements over time. The AI based system 100 checks whether the patient is present in frame or not or and if the video input has ended. If the patient is not present in the frame then the AI based system 100 checks the next frame. If the video input has ended, the AI based system 100 stops.

In some examples, the AI based system 100 comprises AI based computer vision techniques including object tracking, motion detection, object identification, and optical flow may be employed to determine medication adherence. In some embodiments, the AI based computer vision techniques is performed by the AI processing module 106 and the gesture-detection module.

For example, the AI based system 100 could monitor medication adherence through a series of steps such as visual confirmation, hand movement tracking, and audio confirmation. The patient could be asked to show to the camera 108A their hand holding the medication to be swallowed, then video could be captured of the patient moving their hand to their mouth and showing their empty hand to the camera 108A. In some examples, audio confirmation of the patient swallowing may be obtained by the microphone 108B.

In another embodiment, the patient monitoring device 102 is configured to receive the prescription data for the patient, and sends the one or more alerts by the alert module 110 when doses are missed. In some embodiments, the alert module 110 may send a missed dose alert to one or more caregiver. In some embodiments, the alert module 110 may send a missed dose alert to the patient or to the physician. The alert module 110 may notify the caregiver or physician of medication adherence or medication compliance determined by the connected patient monitoring device 102 as a function of sensor data. Further, the alert module 110 provides dose reminders to the patient and compliance the alerts to the patient and the authorized user 118.

In one embodiment herein, the output module 112 is configured to transmit the schedule data, and the generated insights and predictive analytics to at least one of the authorized user 118 and the patient. In another embodiment, the output module 112 comprises a display screen 112A of the patient monitoring device 102.

In one embodiment herein, the identification module 130 is configured to identify one or more medications that are dispensed to the patient by the patient monitoring device 102. The medications comprise, but are not limited to, pills, doses of liquids, injectables, and the like, that may be automatically dispensed to the patient. The identification module 130 is capable of recognizing the different medications and keeping track of when they are dispensed to the patient by the patient monitoring device 102. In one embodiment herein, the patient monitoring device 102 is configured to automatically dispense medications to the patient. The patient monitoring device 102 can dispense the medication at specific times or intervals, according to the instructions provided by the patient's healthcare provider. By automating the process of medication dispensing, the AI based system 100 can assist to ensure that the patient is taking their medications on time and according to the correct dosage, which is important for maintaining optimal health outcomes.

In one embodiment, the AI based system 100 is well-suited for managing medication adherence, which is a critical aspect of patient care. By identifying and tracking medications that are dispensed to the patient, the AI based system 100 can help healthcare providers to remotely monitor patient compliance with medication protocols and provide early interventions if necessary. This could lead to improved patient outcomes and reduced healthcare costs, making the system a valuable resource for both patients and healthcare providers.

In one embodiment herein, the patient monitoring device 102 comprises at least one capturing unit. The capturing unit include, but not limited to, a camera, an image sensor, and other sensors. The capturing unit is adapted to input video and image data of dispensed medications. The input video and image data is transmitted to the identification module 130. The identification module 130 could be a trained module that is trained with various medications for accurately identifying the dispensed medicine using the AI processing module 106. Further, the identification module 130 is in communication with the server 116, where the patient profile 120 is stored.

When the medicine is dispensed, the capturing unit captures the video and image data which is then analyzed by the identification module 130 to accurately identify the medicine. After identification, the identification module 130 accesses the patient profile 120 to ensure that the correct medication is dispensed. If, the dispensed medicine is correct, an affirmative alert is provided through the output module 112, thereby indicating that the patient can consume the dispensed medicine. If the dispensed medicine is incorrect, a warning alert is sent through the output module 112, thereby indicating that the dispensed medicine should not be consumed.

In another embodiment, the AI processing module 106 is configured to analyze the patient's voice to identify one or more vocal biomarkers that are indicative of a patient's health state, and a patient's emotional state. In one embodiment herein, the vocal biomarkers comprise, but are not limited to, pitch, tremor, and speech patterns. The AI processing module 106 is configured to correlate the vocal biomarkers with a symptom database using a symptom-checking module to identify potential symptoms of the patient based on the analyzed vocal biomarkers. In one embodiment, the symptom-checking module is a pre-trained module that is configured for symptom identification.

In one embodiment herein, the alert module 110 is configured to transmit the one or more alerts to the authorized user about the identified potential symptoms of the patient. Further, the output module 112 is configured to provide a vocal aid to the patient based on the identified potential symptoms. In one embodiment herein, the vocal aid is selected from a group consisting of feedback on the patient's emotional state and prompts to seek professional help or access mental health resources. In another embodiment, the output module 112 is configured to transmit the schedule data to at least one of the authorized user 118 and the patient. In another embodiment, the output module 112 connected to the display screen 112A or a speaker 112B of the patient monitoring device 102.

In one embodiment, the patient monitoring device 102 comprises the communication module 132. The communication module 132 is configured to enable the patient to communicate with the authorized user 118 through either a voice call or a video call via the display screen 112A of the patient monitoring device 102.

In another embodiment, communication could be possible between a physician, remote caregiver, pharmacist, and the patients or local caregivers through a web application or a mobile application installed on the patients', physicians', and remote caregivers' mobile devices. In some exemplary scenarios, if a patient fails to respond to a timed dispense of medication within a time window specified by the pharmacist and/or physician/remote caregiver, a missed dose alert is sent to the patient.

In another embodiment, the authorized user 118 and the patient are allowed to customize the schedule data through the user device 122.

In another embodiment, the patient may employ a wearable device that is configured with sensors to measure the real-time patient vital sign data. In another embodiment, the wearable device is communicatively and operably coupled with the network 114 and sends the measured real-time patient vital sign data to the patient monitoring device 102 via the network 114. The measured real-time patient vital sign data may also be sent to the authorized user 118 through a web application accessed by a desktop computer. The measured real-time patient vital sign data may also be sent to a pharmacy server, a physician server, a caregiver device, a patient's user device, and patient-selected third parties through the network 114.

Moreover, the real-time patient vital sign data may also be collected at the server 116. In some embodiments, the user device 122 is configured with a mobile app (“software application”) adapted to assist the authorized user 118 in treatment of the patient. In the some embodiments, the authorized user 118 diagnoses the illness of the patient and determines a prescription protocol to treat the patient illness. The mobile app of the user device 122 creates a prescription, based on the prescription protocol determined by the authorized user 118. Alternatively, the prescription may be created/sent by the authorized user 118 through a web portal. In another embodiment, the user device 122 includes a caregiver device, and a patient's user device.

Referring to FIGS. 1B-1C, the patient monitoring device 102 is configured to deliver a dose to the patient based on the prescription data. The patient monitoring device 102 comprises components which may include the display screen 112A, one or more camera 108A, the microphone 108B, the speaker 112B, a power source 144, the processor 126 comprises a jetson nano unit 152, a cellular communication module 146, one or more sensors (136, 138), one or more visual indicators (not shown), a servo motor (not shown), a custom PCB board 148 with control software embodied therein, a brush-less DC motors 142, a chamber 160, a cup 158, and one or more outlet 154, and a glucometer slot 156.

In another embodiment, the jetson nano unit 152 is configured for controlling medication scheduling, dispensing mechanisms, and potentially interfacing with refill services or caregivers. The cellular communication module 146 is configured to allow the patient monitoring device 102 to connect to a cellular network. This is used for remote monitoring by caregivers, medication refill alerts, or two-way communication features. The cellular communication module 146 is GNSS module LTE CAT4.

An IR presence sensor 136 detects the presences of the cup 158. In another embodiment, the IR presence sensor 136 is configured to detect if someone has retrieved the dispensed medication, and sending a notification if the medication isn't retrieved within a certain timeframe. Additional components may include a limit switch to register when the cup 158 comprising dispensed medication is removed from the chamber 160.

The visual indicators are illuminating lights or RGB LED that are utilized for various visual cues, such as indicating when a medication is ready, low battery warnings, or potential dispensing errors. The servo motor is well-suited for precise movements. In the patient monitoring device 102, it could be used to control a dispensing mechanism, such as a carousel or pill-pusher, to deliver the correct medication and dosage. The glucometer slot 156 is a designated opening where a test strip is inserted. In another embodiment, the brush-less DC motors 142 is configured to control the dispensing mechanism.

The patient monitoring device 102 comprises an openable cabin door 150. The openable cabin door 150 is configured to be opened to access a wheel slot unit 140. The wheel slot unit 140 is configured with one or more slots for storing the medications. The wheel slot unit 140 is secured within the patient monitoring device 102.

In one embodiment herein, the AI based system 100 comprises a plurality of holders 151 mounted on both sides of the patient monitoring device 102. The holders 151 are configured to securely hold plurality of medicines. In one embodiment herein, the medicines include, but not limited to, tablets or pills, powder medicines, granule medicines, capsule medicines, medication bottles, medication container, and spray medication bottles.

For example, the holders 151 are configured to store plurality of medication bottles. The holders 151 are in communication with the processor 126. If the patient or user misses his medication then the processor 126 alerts the user or the guardian by sending notifications to the user device 122 and also by illuminating the visual indicators. In one embodiment herein, if the user grabs the medicine from the holders 151, then the processor 126 alerts the guardian by sending the notification to the user device 122.

In one embodiment, each holder 151 is configured holding at least one medicine. In one embodiment herein, the each holder 151 is configured to hold a medication bottle or a medication container. Each holder 151 is configured with at least one sensor (not shown) that is configured for, but not limited to, an inventory control, a dosage verification, and detecting empty or missing the medicine. The sensor comprises, but is not limited to, a load cell, and a piezoresistive sensor, thereof.

In one exemplary embodiment, the sensor comprises a load cell. The load cell is disposed at bottom of the holders 151. In one embodiment, each holder 151 comprises a compartment having various shapes and sizes, and the load cell is placed in the compartment such that when a medication bottle is placed on the holder 151, its weight presses down on the load cell. The load cell converts this pressure into an electrical signal that can be measured. The electrical signal is translated into the weight of the medication bottle by the processor 126.

In one embodiment, the AI-based system 100 monitors the weight of medication bottles in order to track the amount of medicine remaining in each medication bottle. This ensures that the correct amount of medicine is consumed by the patient and also detects empty or missing bottles with the assistance of the load cell.

The camera 108A of the monitoring module 108 is configured to capture either video or image data of the patient during medication consumption activity. The gesture-detection module analyzes the captured video or image data to detect any abnormal actions related to medication adherence. These abnormal actions may indicate potential non-adherence behaviors. The gesture-detection module compares the captured data with the schedule data to identify the timing and frequency of medication adherence events, which helps in identifying potential medication non-adherence, such as missed doses or potential overdoses. The authorized user 118 is then notified of these potential issues through alerts or reminders in the form of audios, videos, notifications, or calls.

In another embodiment, the camera 108A may capture time stamped images or video data of patient actions after the medication bottle is removed from the holder 151. Such cameras may include controllers that are electrically and communicatively connected to the processor 126 of the patient monitoring device 102. The captured time stamped images or video data is analyzed through the AI-based computer vision or the gesture-detection module or both. This process enables the identification of precise timing and frequency of medication adherence events, which is vital in detecting potential medication non-adherence scenarios such as missed doses or potential overdoses. By doing so, potential medication non-adherence scenarios such as missed doses or potential overdoses can be pinpointed with greater accuracy. This aids in identifying non-adherence events. The AI-based computer vision and the gesture-detection module can also be trained to recognize specific gestures associated with medication adherence, such as swallowing pills, syrup consumption levels, and thereof. This further enhances the accuracy of the AI based system 100 and allows for a more comprehensive approach to medication management.

In case of the potential medication non-adherence detection, the alert module 110 generates a real-time audible alert to notify the patient, and send real-time notifications regarding the lack of adherence to one or more authorized users. The authorized user 118 include a pharmacy server, a physician server, a caregiver device, the patient's user device, and patient-selected third parties. The cameras may also provide network enabled video feed, such as video conferencing between the patient and caregiver, and a physician, pharmacy, thereof.

The patient monitoring device 102 disclosed herein may include illuminating lights that illuminate a portion of the outer surface of the holder 151 or speakers 112B to provide communication with the patient to the authorized user 118.

In some embodiments, the patient monitoring device 102 may use object-tracking methods to track and record the patient's hand motions as they ingest the medication. This includes monitoring the movement of the patient's hand as they take a cup of liquid from a table, lift it to their mouth, swallow the liquid, and return the cup to the table. By comparing the patient's hand motion to a previously established motion emblematic of a successful dose event, the dispenser can establish medication adherence.

In another embodiment, the user device 122 is in communication with the patient monitoring device 102 through the LoRA modules. For example, the user device 122 is configured to transfer an encrypted data through a user LoRA module. The edge node in the server 116 is configured to receive the encrypted data from the user device 122 via a system LoRA module. The received encrypted data is decrypted and stored in a database 134. Similar operation is performed when an encrypted data is transferred from the server 116 to the user device 122. In some embodiments, each LoRA module is a LoRaWAN backhaul architecture

In another embodiment, the patient monitoring device 102 is equipped with the LoRaWAN backhaul architecture to ensure uninterrupted connectivity with the server 116 in case of disruptions in WIFI, LTE/5G WAN links. This technology provides a reliable and robust means of establishing connectivity through a low-power, wide-area network in the event of a network outage. The LoRaWAN backhaul architecture serves as an effective redundancy mechanism, ensuring that critical patient data is transmitted securely and without delay.

In one exemplary embodiment herein, the patient monitoring device 102 is in communication with the server 116 through the network 114 or LoRaWAN. The patient monitoring device 102 is configured to continuously and remotely monitor the patient through the camera 108A, and the microphone 112B. The camera 108A, and the microphone 112B capture video data and audio data of the patient in real-time. The camera 108A includes, but not limited to, an AI camera.

The video data is processed by the AI processing module 106, and the gesture-detection module to perform a face detection operation, a face recognition operation, a pose estimation operation, and action detection. The audio data is processed by the AI processing module 106 to extract vocal biomarkers that are indicative of a patient's health state, and a patient's emotional state, and recognize command. The video data and the audio data are converted into a Meta data by the AI processing module 106. The Meta data is processed to provide appropriate response to the patient and the authorized user 118.

In another embodiment, the LoRaWAN backhaul architecture incorporates two layers of cryptography to guarantee security. The first layer entails a unique 128-bit Network Session Key that is mutually shared between end devices and the server 116. Herein, the end devices comprises, but are not limited to, one or more patient monitoring devices 102, and one or more user devices 122. The second layer involves a unique 128-bit Application Session Key (AppSKey) that is shared end-to-end at an application level. The server 116 is responsible for providing authentication and packet integrity using the Advanced Encryption Standard (AES) algorithms, while the application server is responsible for handling end-to-end encryption. By providing these two levels of security, it becomes feasible to implement ‘multi-tenant’ shared networks without compromising the users' data privacy.

In another embodiment, the keys may be activated through the process of Activated by Personalization (ABP) during the production phase or commissioning, or alternatively, through Over-The-Air Activation (OTAA) in the field. The OTAA approach permits devices to be re-keyed if required, thereby adding greater flexibility to the process.

In an exemplary embodiment, the patient monitoring device 102 is in communication with respective user devices 122 through the LoRA modules. From the user devices 122, the data is visualised through a web application or mobile application or can be synchronized with server 116 or database 134.

In another exemplary embodiment, multiple patient monitoring devices 102 are in communication with at least one LoRa base station. Each LoRa base station is in communication with the database 134 through the network 114. From the network 114, the data is transferred to the server 116, where it is then stored.

In some embodiments, the storage is located on disks attached physically to a host computer. This storage is well-suited for frequently changing temporary data, like temporary workspaces (scratch data), caches, and buffers. By default, files on these disks are not encrypted. Encryption is available to protect data confidentiality. When enabled, all data and its details such as metadata are encrypted at rest using a strong industry standard. Encryption and decryption happen automatically without needing any user intervention. An application programming interface (API) is used to obtain user permission for data access.

Once granted, connection details are secured using temporary tokens that the web application or mobile application needs to refresh regularly. To ensure a secure connection, the app relies on a unique and reliable client ID and secret. All communication over the internet is encrypted using the industry standard HTTPS/TLS. Similar to data encryption, communication encryption and decryption also happen automatically.

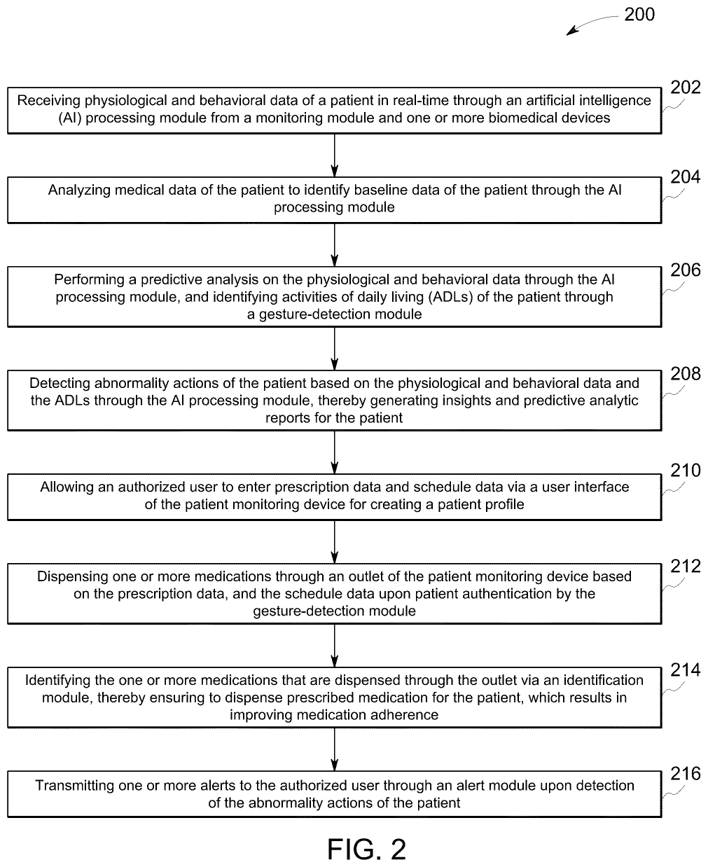

FIG. 2 refers to a flowchart 200 of a method for monitoring patient's health in real-time and facilitate in medication adherence for users using the patient monitoring device 102. The method comprises, receiving physiological and behavioral data of the patient in real-time through the AI processing module 106 from the monitoring module 108 and the biomedical devices 124, as depicted at step 202. The method comprises, analyzing medical data of the patient to identify baseline data of the patient through the AI processing module 106 as depicted at step 204. The method comprises, performing a predictive analysis on the physiological and behavioral data through the AI processing module 106, and identifying activities of daily living (ADLs) of the patient through the gesture-detection module as depicted at step 206. The method comprises, detecting abnormality actions of the patient based on the physiological and behavioral data and the ADLs through the AI processing module 106, thereby generating insights and predictive analytic reports for the patient, as depicted at step 208.

The method comprises, allowing the authorized person to enter prescription data and schedule data via the user interface of the patient monitoring device 102 for creating a patient profile 120, as depicted at step 210. The method comprises, dispensing the medications through the outlet of the patient monitoring device 102 based on the prescription data, and the schedule data upon patient authentication by the gesture-detection module, as depicted at step 212. The method comprises, identifying the one or more medications that are dispensed through the outlet via the identification module 130 as depicted at step 214. This ensures to dispense prescribed medication for the patient, which results in improving medication adherence. The method comprises, transmitting one or more alerts to the authorized user through the alert module 110 upon detection of the abnormality actions of the patients as depicted at step 216.

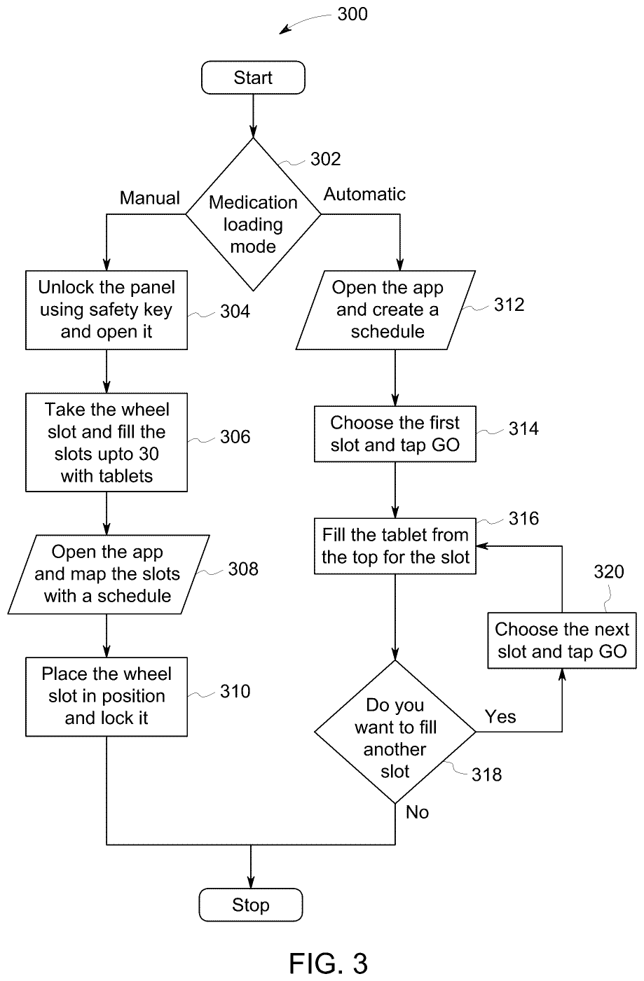

FIG. 3 refers to a flowchart 300 of a method for medication refilling into the patient monitoring device 102. The patient monitoring device 102 provides two mode of medication refiling such a manual mode and an automatic mode. At step 302, mode of medication refiling is selected the authorized user 118.

In manual mode, the medication panel is opened through a safety key by the authorized user 118, as depicted at step 304. At step 306, the wheel slot unit 140 is taken out of the patient monitoring device 102, and each slot of the wheel slot unit 140 is refilled with an appropriate medication in prescribed doses. After refilling the slots, the authorized user 118 map the slots with schedule through the user device 122, as depicted at step 308. At step 310, the wheel slot unit 140 is placed inside the patient monitoring device 102 and locked with the safety key.

In automatic mode, the authorized user 118 creates a schedule through the user device 122, as depicted at step 312. At step 314, the authorized user 118 selects a first slot of the wheel slot unit 140 through the user device 122 and start medication refilling operation. Each slot is filled with the medication, at step 316. After refilling the first slot, the AI based system 100 inquires whether another slot is needed to be filled, as depicted at step 318. If no, the medication refilling operation is stopped. Else, the authorized user 118 selects next slot of the wheel slot unit 140 through the user device 122 and start medication refilling operation, as depicted at step 320. This step is repeated till each slot of the wheel slot unit 140 is filled.

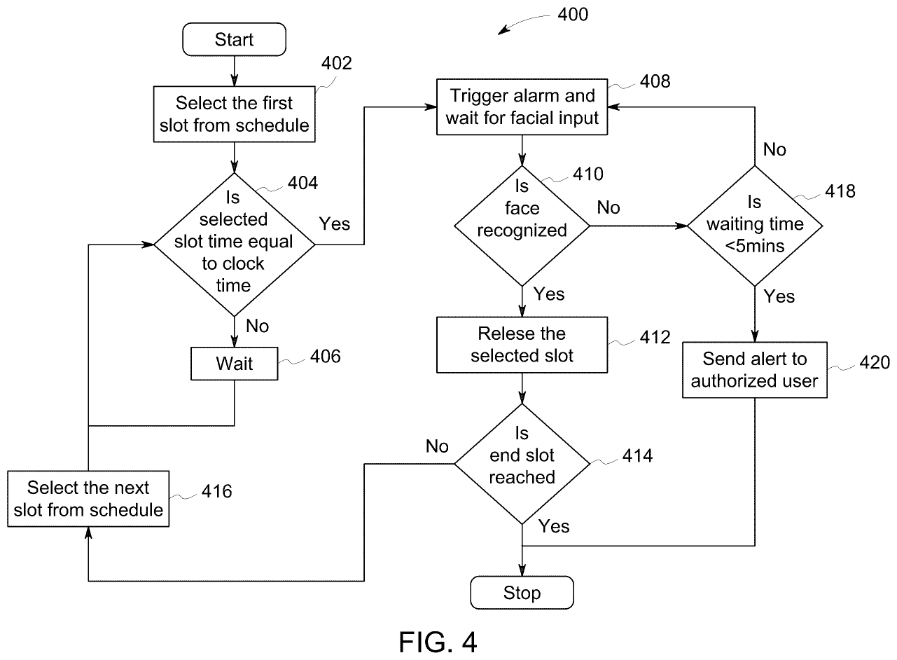

FIG. 4 refers to a flowchart 400 of a method for medication dispensing from the patient monitoring device 102. At step 402, the patient selects a first slot through the display unit 112A based on the schedule data. At step 404, the control unit verifies whether a real-time slot selection time matches with a scheduled slot selection time. In one embodiment, the real-time slot selection time is the time when the first slot is selected by the patient to dispense the medication, while the scheduled slot selection time is the time when the first slot is supposed to be selected as per the schedule data.

If the real-time slot selection time does not match the scheduled slot selection time, the patient monitoring device 102 will provide an alert or notification through the output module 112. The alert will request the patient to wait to select the first slot at the scheduled time, based on the patient's schedule, as shown in step 406.

Else, an alert is generated through the output module 112, requesting the patient to stay still for at least 5 min to enable the camera 108A to capture the patient image. The patient image is useful for a patient authentication, as depicted at step 408.

At step 410, the gesture-detection module utilizes the facial recognition model for patient authentication. This process involves facial biometric feature identification by comparing the patient's real-time facial geometry against a previously captured reference image. The geometry of facial features comprise, but are not limited to, displacement and structure of eye sockets, nasal bridge, or mouth or lip geometries. By comparing these key geometric features, the system can achieve a high level of accuracy in patient identification

If the patient authentication fails, i.e. the AI based system 100 fails to identify the patient. Then, the processor check whether the patient stood still for less than 5 min, at step 418. If the patient stayed still for more than 5 min, then again the camera 108A is activated to take the patient image. Else, one or more alerts are provided to the authorized user 118, at step 420.

If the patient authentication is succeeded, i.e. the AI based system 100 has identified the patient. Then, the selected slot is releases the medication from the outlet, as shown at step 412. Later, the processor will ask the patient if the end of the slot is reached. If end of the slot is not reached, then next slot is selected from the schedule data, as shown at step 416. Else, the patient monitoring device 102 will stop the medication dispensing process.

In another embodiment, the AI based system 100 enables data flow with the network 114 and without the network 114. In with network 114 scenario, the data flow starts with a “Start Read/Write”. This could represent any action that initiates data transfer, such as opening a file or starting an application. The data is then transferred through the network 114. The AI based system 100 asks if the patient monitoring device 102 “is able to communicate to the network 114”. If yes, and if there is local data present, the data is updated. If there is no local data present, or if the patient monitoring device 102 cannot communicate with the network 114, a timeout occurs.

In the without network 114 scenario, the data transfer starts the same way, but instead of traveling through the network 114, the data transmits through a local storage media, likely a hard drive or USB. The AI based system 100 then asks if the patient monitoring device 102 is able to communicate with LoRa, a low-power wide-area network technology. If yes, and if there is local data present, the data is updated. If there is no local data present, or if the patient monitoring device 102 cannot communicate with LoRa, a timeout occurs.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A system for monitoring patient's health in real-time, comprising:
a patient monitoring device having a processor and a memory for storing one or more instructions executable by the processor, wherein the patient monitoring device is in communication with a server via a network,
wherein the processor is configured to perform one or more operations include:
receiving physiological and behavioral data of a patient in real-time through an artificial intelligence (AI) processing module from a monitoring module and one or more biomedical devices;
analyzing medical data of the patient to identify baseline data of the patient through the AI processing module;
performing a predictive analysis on the physiological and behavioral data through the AI processing module, and identifying activities of daily living (ADLs) of the patient through a gesture-detection module;
detecting abnormality actions of the patient based on the physiological and behavioral data and the ADLs through the AI processing module, thereby generating insights and predictive analytic reports for the patient;
allowing an authorized person to enter prescription data and schedule data via a user interface of the patient monitoring device for creating a patient profile;
dispensing one or more medications through an outlet of the patient monitoring device based on the prescription data, and the schedule data upon patient authentication by the gesture-detection module;
identifying the one or more medications that are dispensed through the outlet via an identification module, thereby ensuring to dispense prescribed medication for the patient, which results in improving medication adherence; and
transmitting one or more alerts to the authorized user through an alert module upon detection of the abnormality actions of the patients.

2. The system of claim 1, wherein the identification module is adapted to receive at least one of video and image data of one or more dispensed medications from at least one capturing unit, thereby accurately identifying the dispensed medicine using the AI processing module.

3. The system of claim 1, wherein the monitoring module is configured to capture real-time images or videos of the patient, and transmit the captured real-time images to the AI processing module to confirm the identity of the patient.

4. The system of claim 1, wherein the gesture-detection module is configured to perform predictive analysis to detect the ADLs based on the physiological and behavioral data of the patient, wherein the gesture-detection module is trained with various physiological and behavioral parameters of multiple patients, wherein the physiological and behavioral data of the patient comprises a real-time patient vital sign data, facial expressions, postures, gestures, limb positions, hand positions, reflexes, qualities of a patient's voice, and intensity.

5. The system of claim 1, wherein the AI processing module is further configured to detect patient's abnormality actions based on deviation in the physiological and behavioral data of the patient with respect to the baseline data.

6. The system of claim 1, wherein the monitoring module is configured to continuously track the physiological and behavioral data of the patient remotely in real-time, wherein the one or more biomedical devices are configured to communicate with the patient monitoring device via the network ensuring seamless, real-time data tracking of the physiological and behavioral data of the patient.

7. The system of claim 1, wherein the monitoring module is configured to track the schedule data, wherein the schedule data comprises at least one of medication dosage, medication administration times, medication name, frequency of medication administration, route of administration, exercise periods, type of exercise, time duration for the exercise, and intensity of the exercise.

8. The system of claim 1, wherein the alert module is further configured to provide one or more alerts and notifications for refiling the medication within the patient monitoring device, wherein the alert module is configured to provide the alerts or reminders in a form of at least one of audios, videos, notifications, and calls for missed medications and upcoming exercise sessions.

9. The system of claim 1, wherein the patient monitoring device comprises an output module that is adapted to transmit the schedule data and the generated insights and predictive analytics to the authorized user and the patient.

10. The system of claim 1, wherein the patient monitoring device comprises a plurality of holders that are mounted on both sides of the patient monitoring device, wherein each holder is configured holding at least one medicine, wherein each holder is configured with at least one sensor that is configured for at least one of an inventory control, a dosage verification, and detecting empty or missing the medicine.

11. The system of claim 1, wherein the monitoring module is configured to capture either video or image data of the patient during medication consumption activity, wherein the captured video or image data is analyzed by the gesture-detection module to detect the abnormality actions that is associated with medication adherence, wherein the abnormality actions are indicative of potential non-adherence behaviors.

12. The system of claim 11, wherein the gesture-detection module is configured to compare the captured video or image data with the schedule data to identify the timing and frequency of medication adherence events to identify the potential medication non-adherence, such as missed doses or potential overdoses, thereby provide the alerts or reminders in a form of at least one of audios, videos, notifications, and calls to the authorized person.

13. A patient monitoring device for monitoring patient's health in real-time, comprising:
a processor and a memory for storing one or more instructions executable by the processor, wherein the patient monitoring device is in communication with a server via a network;
an artificial intelligence (AI) processing module configured to receive physiological and behavioral data of a patient in real-time from a monitoring module and one or more biomedical devices, wherein said AI processing module is adapted to analyzing medical data of the patient to remotely identify baseline data of the patient;
a gesture-detection module configured to perform a predictive analysis on the physiological and behavioral data and identify activities of daily living (ADLs) of the patient,
wherein said gesture-detection module is in communication with said AI processing module for transferring the predictive analysis on the physiological and behavioral data the activities of daily living of the patient, thereby detecting abnormality actions of the patient and generating insights and predictive analytic reports for the patient;
a user interface configured to allow an authorized person to enter prescription data and schedule data for creating a patient profile;
at least one outlet configured to dispense one or more medications based on the prescription data, and the schedule data upon patient authentication by said gesture-detection module;
an identification module configured to identify the one or more medications that are dispensed through the outlet, thereby ensuring to dispense prescribed medication for the patient, which results in improving medication adherence;
an alert module configured to transfer one or more alerts to the authorized user upon detection of the abnormality actions of the patients; and
an output module configured to transmit the schedule data and the generated insights and predictive analytics to the authorized user and the patient.

14. The patient monitoring device of claim 13, wherein the alert module is further configured to provide one or more alerts and notifications for refiling the medication within the patient monitoring device.

15. The patient monitoring device of claim 13, wherein the monitoring module is configured to capture real-time images of the patient, and transmit the captured real-time images to the AI processing module to confirm the identity of the patient, wherein the monitoring module is configured to continuously track the physiological and behavioral data of the patient in real-time.

16. The patient monitoring device of claim 13, wherein the gesture-detection module is configured to perform predictive analysis to detect the ADLs based on the physiological and behavioral data of the patient, wherein the gesture-detection module is trained with various physiological and behavioral parameters of multiple patients.

17. The patient monitoring device of claim 13, wherein the AI processing module is further configured to detect patient's abnormality actions based on deviation in the physiological and behavioral data of the patient with respect to the baseline data.

18. The patient monitoring device of claim 13, wherein the physiological and behavioral data of the patient comprises a real-time patient vital sign data, facial expressions, postures, gestures, limb positions, hand positions, reflexes, qualities of a patient's voice, and intensity, wherein the one or more biomedical devices are configured to communicate with the patient monitoring device via the network.

19. The patient monitoring device of claim 13, wherein the monitoring module is configured to track the schedule data, wherein the schedule data comprises at least one of medication dosage, medication administration times, medication name, frequency of medication administration, route of administration, exercise periods, type of exercise, time duration for the exercise, and intensity of the exercise.

20. A method for monitoring patient's health in real-time and facilitate in medication adherence for users using a patient monitoring device, comprising:
receiving physiological and behavioral data of a patient in real-time through an artificial intelligence (AI) processing module from a monitoring module and one or more biomedical devices;
analyzing medical data of the patient to identify baseline data of the patient through the AI processing module;
performing a predictive analysis on the physiological and behavioral data through the AI processing module, and identifying activities of daily living (ADLs) of the patient through a gesture-detection module;
detecting abnormality actions of the patient based on the physiological and behavioral data and the ADLs through the AI processing module, thereby generating insights and predictive analytic reports for the patient;
allowing an authorized person to enter prescription data and schedule data via a user interface of the patient monitoring device for creating a patient profile;
dispensing one or more medications through an outlet of the patient monitoring device based on the prescription data, and the schedule data upon patient authentication by the gesture-detection module;
identifying the one or more medications that are dispensed through the outlet via an identification module, thereby ensuring to dispense prescribed medication for the patient, which results in improving medication adherence; and
transmitting one or more alerts to the authorized user through an alert module upon detection of the abnormality actions of the patients.
